(19) United States
(12) Patent Application Publication
(10) Pub. No.: US 2020/0121943 A1
(43) Pub. Date: Apr. 23, 2020

(54) UVB LIGHT THERAPY FOR IMMUNE DISORDERS

(71) Applicant: LUMA THERAPEUTICS, INC., Millbrae, CA (US)

(72) Inventors: Evan R. ANDERSON, Redwood City, CA (US); John MACMAHON, Exeter, NH (US); Christopher Steven PELL, San Francisco, CA (US)

(21) Appl. No.: 16/477,798

(22) PCT Filed: Jan. 16, 2018
(86) PCT No.: PCT/US18/13865

§ 371 Date: Jul. 12, 2019

Related U.S. Application Data
(60) Provisional application No. 62/446,321, filed on Jan. 13, 2017.

(51) Int. Cl. A61N 5/06

(52) U.S. Cl. CPC A61N 5/0613; A61N 5/0601; A61N 2005/0661; A61N 2005/0627; A61N 2005/0602; A61N 2005/0643; A61N 2005/063

(57) ABSTRACT

Methods and apparatuses for immunosuppression and/or immunomodulation for the treatment of an improper immune response with the use of UV light. In particular, described herein are methods and apparatuses for the treatment of inflammatory disorders using UVB light at appropriate intensities, durations and (internal) body regions in order to effect substantial and reliably treatment.

### CROSS REFERENCE TO RELATED APPLICATIONS

This patent application claims priority to U.S. provisional patent application No. 62/446,321, filed on Jan. 13, 2017, titled “UVB LIGHT THERAPY FOR IMMUNE DISORDERS.” This application is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Method and apparatuses for immunosuppression and or immunomodulation in the treatment of various diseases and conditions found in humans using UVB light is described herein.

### BACKGROUND

The immune system is known to play a role in numerous disease state and human conditions such as inflammatory skin disease (psoriasis, vitiligo, dermatitis, etc.), Atherscleroisis, Autoimmune disease (RA, lupus, IBD, Multiple Sclerosis (MS), Chrohns, Guilliane-Barre & CIDP, Graves, Myasthenis Gravis, Vasculitis, Cancer, transplant and implant rejection, infection, allergies and mental disorders. As a host defense system, the immune system protects an organism against various diseases and can evolve over time to provide immunity to various pathogens. In some instances, the immune system may become hyper-reactive and attack the host organism, causing various diseases and conditions. In other instances, the immune system may become hyper-reactive to non-threatening molecules or organisms, eliciting an improper immune response and seen in allergies.

Ultraviolet B (UVB) light exposure of the skin has been suggested to suppress the immune response through a complex cascade of events. The immune suppression may be systemic, and has been shown to aid in acceptance of transplanted organs and to treat inflammatory skin disease such as psoriasis, when the light is applied externally to the skin. The overwhelming research on immune suppression by UVB light describes only the use of externally applied UV light, or in some instances, application through a natural orifice, in order to generate a systemic response. Unfortunately, this approach is limited by a high level of skin variation, the need for repeated exposure to large areas of the skin in multiple treatments for immunomodulation and immune suppression and the natural photoadaptation that takes place upon multiple doses of light.

It would be beneficial to provide therapies and methods of delivering them that may reliably (and implantably) be used to treat patients for inflammatory disorders. Described herein are methods and apparatuses that may address these needs.

### SUMMARY OF THE DISCLOSURE

Described herein are methods and apparatuses (including systems, devices, etc.) for immunosuppression and/or immunomodulation for the treatment of an improper immune response with the use of UV light. Specifically, the methods and apparatuses described herein may use UVB light at appropriate intensities, durations and (internal) body regions in order to effect substantial and reliably treatment. For example, described herein are apparatuses and methods for systemic immunosuppression and/or immunomodulation by the application of UVB light to the lymphatic system from an internal location such as a lymph node or duct. Direct UVB exposure of the immune system (e.g., from an implanted or internal location) may dramatically and unexpectedly enhance the effect of UVB light, potentiating the effects and making them more consistent and easier to deliver in a controlled manner. In addition, direct UVB exposure of the immune system may avoid the photoadaptation response of the skin that may otherwise decrease the effects of UVB light and/or make them less repeatable and reliable when treating immunological disorders.

Also described herein are methods and apparatuses for internal UVB therapies that may include an integrated feedback (e.g., biofeedback) mechanism, allowing tuning of the immune system. The feedback mechanisms may include organic, in vivo or ex vivo feedback. For example, the feedback may provide a preventive method to delivered and modulate a region of a body in anticipation of implants to reduce graph vs. host disease.

In general, the methods and apparatuses described herein are implants that may delivery UV light (e.g., UVB light) to a target tissue or biological material while protecting non-target tissues. For example, described herein are enclosed or semi-enclosed light chambers that may be implanted into the body so that UV light may be delivered to target tissue(s) (e.g., cells, such as immune cells, etc.) within the light chambers without irradiating tissue outside of the light chamber.

For example, described herein are implantable apparatuses for applying ultraviolet (e.g., UVA, UVB, both, etc.) illumination in a contained manner within a patient's body, the apparatus comprising: a frame having a lumen forming a channel for passing a biological material; a UV emitter within the channel; and a UV driver coupled to the UV emitter, wherein the UV driver includes a power source and a controller to control UV emission from the UV emitter, so that the UV light illuminates the biological material passing through the channel.

In some variations the light chamber is a frame that forms a channel or passage (e.g., having a lumen forming a channel) therethrough. An implantable apparatus for applying ultraviolet (e.g., UVA, UVB, both, etc.) illumination in a contained manner within a patient's body may include: a frame having a lumen forming a channel for passing a biological material; a UV emitter within the channel; a UV driver coupled to the UV emitter, wherein the UV driver includes a power source and a controller to control UV emission from the UV emitter; and a UV reflective or absorptive surface in or around the channel, wherein the UV reflective or absorptive surface is configured to block or reflect UV light from the UV emitter, so that the UV light illuminates the biological material passing through the channel but not laterally adjacent to the channel.

The frame may be any appropriate frame, and may be an expandable frame (e.g., an expandable stent). The frame may be rigid or non-rigid, and may act as a support. The frame may include one or more struts, beams, or the like.

The UV emitter may be any appropriate UV emitter, and may include or may be connected to a UV source, such as an LED or laser. For example, the UV emitter may comprise a fiber optic (an optical fiber). The fiber optic may be a standard fiber optic (e.g., a flexible fiber with a UV transparent core, such as a glass core, through which light signals can be sent with very little loss of strength). The fiber optic may be adapted for UV transmission, having a low loss for wavelengths between 290 to 320 nm. In some variations the UV emitter is a UV LED.

The UV emitter may be mounted within the channel through the frame. For example, the UV emitter may be coupled to the side of the channel. The UV emitter may be mounted in a central region of the channel.

Any of these apparatuses may include a cord (e.g., line, cable, fiber, etc.) extending between the UV driver and the UV emitter. The cord may be flexible or rigid or semi-rigid. For example, the court may include a fiber optic cable, which may be particularly useful when the UV driver include one or more UV light sources. Electrical conductivity may also be achieved with spray-on conductive materials.

The UV driver may include a housing (e.g., made of a biocompatible material) enclosing the power source and the controller. The housing may be implanted and connected to the rest of the device by the cord.

Any of the apparatuses described herein may include a power source (e.g., battery, capacitive power source, etc.). The power source may be rechargeable or regenerative. For example, the power source may comprise a rechargeable battery.

Any appropriate controller may be used. For example, the controller may include circuitry and one or more microprocessors. The controller may include a memory (e.g., one or more registers), a timer, one or more power control circuits, etc. The controller may comprise a microcontroller.

In general, the UV reflective or absorptive surface may comprise a reflective surface within the channel. The reflective surface may be specifically reflective for UV wavelengths (permitting other light wavelengths through). The UV reflective or absorptive surface may be a cover and/or liner. The UV reflective or absorptive surface may be a coating or layer. For example, the UV reflective or absorptive surface may be a tube of material within, on, or over the frame. The UV reflective or absorptive surface may be continuous (e.g., with openings at either ends for material to pass into and out of the channel of the frame, but otherwise closed, prohibiting movement laterally out of the frame).

For example, an implantable apparatus for applying ultraviolet (e.g., UVA, UVB, etc.) illumination in a contained manner within a patient's body may include: an expandable frame having a lumen forming a channel for passing a biological material; a UV emitter within the channel; a UV driver coupled to the UV emitter, wherein the UV driver includes a power source and a controller to control UV emission from the UV emitter within the UV range of 290 to 320 nm; and a UV reflective or absorptive surface in or around the channel, wherein the UV reflective or absorptive surface has a reflective inner surface and is configured to block or reflect UV light from the UV emitter within the channel, so that the UV light illuminates the biological material passing through the channel but not laterally adjacent to the channel of the device.

Also described herein are methods. For example, described herein are methods of applying ultraviolet (e.g., UVB) illumination in a contained manner within a patient's body, the method comprising: turning on a UV emitter that is positioned within a lumen forming a channel through a frame, wherein the frame is implanted into a lumen of a vessel in the patient's body; emitting light from the UV emitter to irradiate biological material passing through from the lumen through the channel; and absorbing or reflecting UV light from a sidewall of the channel to prevent irradiation of a region of the lumen of the vessel that is laterally adjacent to the channel.

Any of these methods may also include implanting or inserting the apparatus into the body. For example, any of these methods may include inserting the frame within the lumen of vessel in the patient's body so that biological fluid passes through the channel. Inserting the frame may comprise inserting the frame proximal to a lymph node.

The apparatuses described herein, and methods of using them, may be used in any tissue in the body, including in particular, body regions having a natural body lumen, such as blood vessels, lymph vessels, lung cavities, and any other vessel, tube, tracts, canals, etc. within the body.

Any of the apparatuses described herein may be self-expanding, so that they may be deployed within the body. For example, any of these apparatuses may be self-expanding to fit into the lumen or the vessel in the patient's body. Thus inserting the apparatus may further comprise allowing the frame to self-expand in the lumen of the vessel in the patient's body.

In any of the method described herein, the method may include turning off the UV emitter after delivering a dose of between 0.01 seconds and 60 seconds (e.g., between 0.01 and 45 seconds, between 0.01 and 30 seconds, between 0.01 and 20 seconds, between 0.01 and 10 seconds, etc.). Multiple doses may be applied, at regular or irregular intervals. For example, multiple doses of the UV light may be delivered by repeating the steps of turning on, emitting light and absorbing or reflecting light at a dose frequency of between 1 and 200 doses/day (e.g., between 1 and 150 doses/day, between 1 and 120 doses/day, between 1 and 100 doses/day, etc.).

Generally, turning on may comprise controlling, by an implanted controller, power delivered to the UV emitter. Emitting light from the UV emitter may comprise emitting light from a fiber optic having a distal end terminating within a lumen of the channel formed through the frame. The fiber optic may be open at the end, forming the emitter; any additional lenses, filters, guides, etc., may be coupled to the fiber optic to modify or direct the light (UV light) emitted, or it may be bare, e.g., exposing the core of the fiber optic.

Also described herein are apparatuses (and methods of using them) including an array of UV emitters on a flexible substrate for emitting light to treat a patient. These arrays may be implanted and may be configured to expose light from just one side. For example, any of these apparatuses may be configured for implantation into the body to emit UV lights on one side of the implanted device, and may block or reflect light from the opposite side. Collimation of the light may be achieved along either one or more axes in the Cartesian, Cylindrical, Spherical, or Polar coordinate systems. These apparatuses may be particularly useful for implanting into or just below the skin.

For example, described herein are implantable apparatuses for applying ultraviolet (e.g., UVB) illumination within a patient's body, the apparatus may comprise: a biocompatible and flexible sheet of substrate; an array of UV emitters on one side of the biocompatible and flexible sheet of substrate, wherein the array of UV emitters are configured to emit light from one side of the sheet; a UV driver, wherein the UV driver includes a power source and a controller to control UV emission from the array of UV emitters; and a cord extending between the UV driver and the array of UV emitters, wherein the cord couples the array of UV emitters to the UV driver.

An implantable apparatus for applying ultraviolet (e.g., UVB) illumination within a patient's body may include: a biocompatible and flexible sheet of substrate; an array of UV emitters (e.g., UVB emitters) on one side of the biocompatible and flexible sheet of substrate, wherein the array of UV emitters are configured to emit light from one side of the sheet but not an opposite side of the sheet; UV driver (e.g., UVB driver), wherein the UV driver includes one or more UV light sources (e.g., UVB light sources) configured to emit light within the UV range of 290 to 320 nm, a power source, and a controller coupled to the one or more UV light sources and the power source and configured to control UVB emission from the array of UV emitters; and a cord comprising a plurality of fiber optics extending between the UV driver and the array of UV emitters, wherein the cord couples the array of UV emitters to the UV driver.

The flexible sheet may generally be bendable so that it can be wrapped around a body region. The flexible sheet may be formed of a biocompatible material. The flexible sheet may be any appropriate thickness, but may generally be thin (e.g., less than 1 cm thick, less than 9 mm thick, less than 8 mm thick, less than 7 mm thick, less than 6 mm thick, less than 5 mm thick, less than 4 mm thick, less than 3 mm thick, less than 2 mm thick, less than 1 mm thick, etc.). In particular, the sheet may be less than 2 mm thick.

The array of UV emitters may be configured to emit light from one side of the sheet but not an opposite side of the sheet. Thus, the sheet may be formed of a material, or may include a coating or layer of a material, that is UV opaque and/or reflective (e.g., UVB opaque and/or reflective). UVB reflective materials are particularly useful.

As mentioned, any of the UV emitters in the array of UV emitters may comprise fiber optics.

The apparatuses described herein may generally include a housing surrounding the UVB driver and enclosing the power source and controller. For example, the housing may be formed of a biocompatible material, and may enclose the controller (e.g., microcontroller, circuitry, memory, etc.), power source (e.g., battery, etc.), and in some variations the light source (e.g., UVB emitting light source such as LED, etc.). The power source may be, e.g., a rechargeable battery. Thus, the UV driver may include a UV light source configured to emit light within the UVB range of 290 to 320 nm. The controller may include a microprocessor. The controller may also include a wireless communications circuit.

In any of the apparatuses described herein, the cord may include a plurality of optical fibers extending between the UV driver and the array of UV emitters.

A method of applying ultraviolet (e.g., UVB) illumination within a patient's body may include: turning on an array of UV emitters that are on a first side of a flexible sheet of substrate that is implanted into a patient's body; emitting light from the array of UV emitters to irradiate biological material facing the first side of the flexible sheet of substrate; and absorbing or reflecting UV light with the sheet of substrate to prevent irradiation of biological material that faces an opposite side of the flexible sheet of substrate.

The method may also include inserting or implanting the apparatus in the body. For example, the method may include implanting the flexible sheet of substrate in the patient's body so that the array of UV emitters face a target body region to be irradiated. Implanting the flexible sheet may include implanting the flexible sheet under the patient's skin.

The method may also include turning off the UV emitter after delivering a dose, e.g., of between 0.01 seconds and 10 seconds. The method may also include applying multiple doses of the UV light (e.g., UVB light) by repeating the steps of turning on, emitting light and absorbing or reflecting light at a dose frequency of, e.g., between 1 and 200 doses/day. Turning on may include controlling, by an implanted controller, power delivered to the array of UV emitters. The controller may control the dose, including the amount of power applied (e.g., the intensity of the light emitted), the duration of illumination, the frequency (if pulsed; lighting may be either continuous or pulsed), and the time between illumination, etc. Emitting light from the array of UV emitters may include emitting light from one or more fiber optic having a distal end terminating in or on the flexible sheet. All of the UV emitters may be illuminated together, or they subsets of UV emitters may be illuminated at different times or for different durations and/or intensities, depending on the dosing.

Also described herein are methods and apparatuses for treating a patient that include enclosed chambers with an array of UV emitters. For example, described herein are implantable apparatus for applying ultraviolet (e.g., UVB) illumination in a contained manner within a patient's body, the apparatus comprising: a frame having a chamber; an array of UV emitters within the chamber; a UV driver coupled to the UV emitter, wherein the UV driver includes a power source and a controller to control UV emission from the UV emitter; and a UV reflective or absorptive surface on or around the chamber, wherein the UV reflective or absorptive surface is configured to block or reflect UV light from the UV emitter, so that the UV light illuminates the biological material passing through the channel but not laterally adjacent to the channel. The frame may be any appropriate frame, including those described above. For example, the frame may be an expandable frame. For example, the frame may be an expandable stent.

The array of UV emitters may comprise one or more fiber optics. The array of UV emitters may be coupled to the side of the chamber. In some variations, the array of UV emitters are mounted in a central region of the chamber.

Any of these apparatus may include a cord extending between the UV driver and the array of UV emitters. The cord may include a fiber optic cable; the UV driver may comprise a UV light source. The UV driver may include a housing enclosing the power source and the controller. The power source may be a rechargeable battery.

In any of these apparatuses, the UV reflective or absorptive surface may comprise a reflective surface (material, coating, layer, etc.) within the channel.

For example, an implantable apparatus for applying ultraviolet B (UVB) illumination in a contained manner within a patient's body may include: a frame having a chamber; an array of UVB emitters within the chamber; a UVB driver, wherein the UVB driver includes one or more UVB light sources configured to emit light within the UVB range of 290 to 320 nm, a power source, and a controller coupled to the one or more UVB light sources and the power source and configured to control UVB emission from the array of UVB emitters; a cord comprising a plurality of fiber optics extending between the UVB driver and the array of UVB emitters, wherein the cord couples the array of UVB emitters to the UVB driver; and a UVB reflective or absorptive surface on or around the chamber, wherein the UVB reflective or absorptive surface is configured to block or reflect UVB light from the UVB emitter, so that the UVB light illuminates the biological material passing through the channel but not laterally adjacent to the channel.

Also described herein are methods of using any of the apparatuses described herein to treat a patient, e.g., for an inflammatory disorder including but not limited to treating a patient for any of the inflammatory disorders described herein. For example, described herein are methods of treating an inflammatory disorder, the method comprising: emitting, from an implanted UVB illumination device comprising an array of UVB light emitters connected to a controller, light within the UVB range of 290 to 320 nm to illuminate a local portion of a patient's body (e.g., the patient's lymphatic system); and applying multiple doses of UVB light for a duration that suppresses the subject's immune response. Although the treatment may be applied in a localized and contained manner, the treatment may be done in a part of the body through which biological materials, such as blood, lymph, etc. pass, thereby treating the material (including fluids) passing through the region. In general, the methods described herein may modulate the subject's immune response, including enhancing or suppressing. In some variations, where explicitly indicated, these methods may be configured to just suppress the immune response.

A method of treating an inflammatory disorder may include: emitting, from an implanted UVB illumination device comprising an array of UVB light emitters connected to a controller, light within the UVB range of 290 to 320 nm to illuminate a local portion of a patient's lymphatic system; wherein the array of UVB light emitters are positioned at the local portion of the patient's lymphatic system and are connected by a flexible cord to a controller that is implanted in a separate region of the patient's body; and applying multiple doses of the UVB light to suppress the subject's immune response.

The local portion of a patient's lymphatic system may comprise one or more of: a lymphatic node and a lymphatic vessel.

As mentioned, the controller may include a housing enclosing a control circuitry, a power source and a UVB light source. The controller may comprise a housing enclosing a controller and a power source.

Applying multiple doses of the UVB light to suppress the subject's immune response may comprise emitting the light for a dose length of between 0.01 seconds and 10 seconds. Applying multiple doses of the UVB light to suppress the subject's immune response may comprise emitting the light at a dose frequency of between 1 and 200 doses/day.

Any of the methods and apparatuses described herein may include using one or more biomarkers to adjust the dosing. For example, a method of treating an inflammatory disorder, the method comprising: emitting, from an implanted UVB illumination device comprising an array of UVB light emitters connected to a controller, a dose of light within the UVB range of 290 to 320 nm to illuminate a local portion of a patient's lymphatic system; and adjusting the dose of light based on one or more biomarker(s) input into the implanted controller; and applying multiple doses of UVB light for a duration that suppresses the subject's immune response.

A biomarker, as used herein, may include a level or amount (or a change in a level or amount) of a biological material, such as a protein, gene (DNA, RNA, mRNA, microRNA, etc.), cell type, antigen, enzyme, antibody, etc. A biomarker may include a level or amount (or a change in a level or amount) of a biological process, such as, e.g., heart rate, respiration rate, blood pressure, perspiration rate, skin conductivity, galvanic skin response, swelling/edema, etc., including unconscious feedback. A biomarker, as used herein, may include patient feedback (e.g., conscious or unconscious feedback), including, but not limited to, estimates of pain and/or discomfort, estimates of sensitivity (skin sensitivity), temperature, estimates or redness, stiffness, etc.

For example, adjusting the dose may include adjusting the power delivered to the UVB light emitter. Adjusting the dose may comprise adjusting the dose based on a level of one or more of: C—reactive protein, cortisol, immune cells, patient feedback, etc. Adjusting the dose may comprise adjusting the dose based on a level of cortisol. Adjusting the dose may comprise adjusting the dose based on a number of immune cells. Adjusting the dose may comprise adjusting the dose based on patient feedback.

The local portion of a patient's lymphatic system may comprise one or more of: a lymphatic node and a lymphatic vessel.

Applying multiple doses of the UVB light to suppress the subject's immune response may comprise emitting the light for a dose length of between 0.01 seconds and 10 seconds. Applying multiple doses of the UVB light to suppress the subject's immune response may comprise emitting the light at a dose frequency of between 1 and 200 doses/day.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a schematic illustration of an example of an implantable UVB apparatus for delivering UVB light within a patient's body. FIG. 1B schematically illustrates another example of an implantable UVB apparatus for delivering UVB light within a patient's body, having a light guide (e.g., fiber optic) connecting to a relatively small UVB emitter (e.g., applicator). Multiple applicators may be connected. FIG. 1C schematically illustrates another example of an implantable UVB apparatus for delivering UVB light within a patient's body, having a plurality of UVB emitters (applicators), each shown with a separate UVB light source connected by wired connection to the controller.

FIGS. 2A-2D illustrate one example of a system for internally illuminating a region of a person's body with UVB light. FIG. 2A shows a portion of a patient's anatomy including a region of the lymphatic system. FIG. 2B is an enlarged view of a region including a lymph duct into which an expandable body (e.g., stent, graft, tube, strut, etc.) has been expanded. FIG. 2C shows an example of the expandable body, which includes an internal surface that is reflective. This internal surface may block or reduce light emitted by the by UVB emitter, preventing damage to the non-target tissues of the vessel, etc. allowing treatment of just the target cells 2025 passing through the duct. Although this example is shown in a lymph duct, it may be implanted into any appropriate body region, including a blood vessel, or any other body tube or vessel. FIG. 2D shows the expandable body of FIG. 2D to which the UVB emitter is attached within the body, localizing the UVB light within the body. Thus in this example, the apparatus includes an at least partially enclosed expandable body (e.g., covered stent) and a UVB emitter within the body (which may be an exposed end of a fiber optic line with or without additional lenses and/or a LED source), connected to a remotely implanted controller (including a power source and control circuitry for driving the emitter). In this example, the apparatus is shown implanted in the patient's thoracic duct (either left, right, or both).

FIGS. 2E-2F illustrates another example of a system including a UVB light with power source and controller shown implanted next to a lymph node. In FIG. 2E, the examples of lymph nodes are shown, including the inguinal node at the groin region. FIG. 2F shows an enlarged view of this region

FIG. 3A is an example of a system with a UVB probe with a power source and controller is placed into the nasal cavity (sinus) and/or mouth to deliver UVB light to the lymphatic system (e.g. lymph nodes in the sinus). FIG. 3B illustrate an example of a system for emitting UVB light as described herein towards a subject's tonsils; in FIG. 3B the system includes a mouthpiece (e.g., configured to fit over the subject's teeth) to which the UVB emitting lights are coupled. FIG. 3C is another example of a system configured to emit UVB light toward the lymph nodes in and around the subject's throat/mouth, configured as a wand or rod with UVB emitting lights at the distal end. FIG. 3D shows an example of an apparatus configured as a nasal insert.

FIG. 4A illustrates another example of a system containing one or more UVB light source(s) with a power source and controller; this system may be implanted (e.g., under the skin, sub-dermally). FIG. 4B illustrates the apparatus implanted under the subject's epidermis on a limb (e.g., leg or arm), though other locations may be used.

FIG. 5 shows one example of a system containing an occlusive dressing that is partially UVB transparent for delivery of UVB light to part of a patient's skin.

FIG. 6A illustrate a human lymphatic system; the implantable UVB emitting apparatuses described herein may be configured for implantation or insertion to apply UVB light to any of these regions of the lymphatic system. For example, FIG. 6B illustrates another example of an implantable system that may be implanted near an organ of the lymphatic system such as the spleen, as shown in FIG. 6B.

FIG. 7A provides detail on the lymph nodes that may be present in a patient's abdomen. FIG. 7B illustrates an example of a UVB emitting apparatus configured as an endoscopic system containing a tip with a water filled balloon and UV LEDs for shining light across the abdominal wall; this apparatus may be used to apply the UVB therapy described herein to any of the lymph nodes of the abdomen. FIG. 7C illustrates a distal end region of an endoscope configured to emit UV (e.g., UVB) light.

FIG. 8 illustrates implantation and UVB stimulation of one or more regions of an internal jugular vein of the left subclavian region. In general, the lymphatic vessels of the central nervous system could be exposed to UVB light as described herein. In FIG. 8, the apparatus, which may be the same or similar to that shown in FIG. 2A, may be used to apply light to any of the lymph nodes/vessels illustrated herein.

FIG. 9 schematically illustrates an example of a system for applying UVB light to the optic nerve.

FIGS. 10A and 10B illustrate the use of a system including a shunt of a vessel (e.g., lymphatic vessel, blood vessel, etc.) that passes through a UVB light source. FIG. 10A shows an anatomical region in which a lymphatic shunt may be placed; FIG. 10B illustrates an example of a lymphatic shunt to which UVB emitting apparatus as described herein is included.

FIG. 11A illustrates a lymphatic system, including lymphatic vessels. FIG. 11B shows an example of an implantable system implanted near an organ of the lymphatic system including a feedback sensor adjacently located to the implant.

FIG. 12 is a table of possible sensors and locations that may be used to provide feedback on any of the apparatuses described herein. Thus, any of the apparatuses described herein may include any of these sensors and may be inserted/implanted in the locations indicated.

FIG. 13A illustrates one example of the use of a UVB light source delivered in combination with a local fluid/drug. The drug may or may not be photoactive to the UV/UVB light. FIG. 13B is similar to that shown in FIG. 13A, but include the use of an isolating occluder.

FIGS. 14A-14C illustrates one example of a system comprising a head and neck covering with internal UV lights, in which the entire head and neck of the wearer may be encompassed by UV lights. FIG. 14A shows a front perspective of a system (configured as a helmet that completely covers the head, including face and neck). FIG. 14B is a schematic of a side view showing the regions of illumination by the light (e.g., UVB light). FIG. 14C shows a schematic of a front view.

FIG. 15 illustrates a table of the dose and frequency for different applications of light that may be applied as described herein, including comparison to skin dosage (e.g., minimal erythemal dose, or MED, based on published data determined from various skin types).

FIG. 16A illustrates a pill that may be swallowed and may outputs UV light (e.g., UVB, and/or UVA, UVC, etc.) within the digestive system, as illustrated in FIG. 16B.

### DETAILED DESCRIPTION

The immune system protects an organism against various diseases and can develop over time to provide immunity to various pathogens but, in some instances, the immune system may become hyper-reactive and attack the host organism, causing various diseases and conditions. Ultraviolet B (UVB) light exposure of the skin may suppress the immune response through a complex cascade of events without serious complications. Complications are anticipated to be significantly reduced when compared to those due to systemic delivery of TNF biologics, such as like Humira, which have been reported to lead to serious and sometimes fatal infections due to bacterial, mycobacterial, invasive fungal, viral, or other opportunistic pathogens.

The immune suppression is systemic and has been shown to treat inflammatory skin disease such as psoriasis. Immune suppression by UVB light has been limited to direct exposure of the skin for its systemic response. This approach is limited by a high level of skin variation and the need for repeated exposure to large areas of the skin in multiple treatments for immunomodulation and immune suppression. Described herein are methods and apparatuses (e.g., systems and devices) for immunosuppression and/or immunomodulation for the treatment of an improper immune response with direct exposure of the immune system to UVB light. By more direct UVB exposure of the immune system, the effect of UVB light may be increased and/or made more consistent and easier to deliver in a controlled manner that UVB exposure of the skin.

In general, described herein are methods and apparatuses for treating a patient, including but not limited to treating a patient having an inflammatory disorder, or a disorder having an inflammatory component, symptom or etiology, particularly one related to activity of the immune system and immune response, by the application of UVB light.

As used herein, UVB light may refer to light having a wavelength within the range of 280-320 nm (e.g., alternatively, between 290 and 320, or between 280 and 315, or between 290 and 315). In particular, the apparatuses and methods described herein may be applied internally, including directly on the lymph system within a patient's body. Thus, described herein are implants and implantable devices for delivering UVB to a body region, including the lymphatic (“lymph”) system or any portion thereof.

In general, the apparatuses described herein may include a power source which may be internal (e.g., battery, capacitive power source, inductive coil(s), etc., or any combination of these) or external, and a light source (e.g., LED, laser, etc.) capable of emitting within the UVB frequency range, either exclusively (e.g., limited to the UVB range) or in some variations in combination with other frequency (e.g., UVA) ranges. These apparatuses and methods of using them may also include one or more applicators (e.g., for delivering the light to a target, particularly internal targets); the applicator may include a lens, reflector, waveguide (including but not limited to a fiber optic), filter, or the like. Any of these method and apparatuses may include circuitry configured for control of the delivery of the light therapy. The circuitry may include control logic including timers and/or scheduling logic. The circuitry may include communications logic and/or circuitry for wired and/or wireless (e.g., Bluetooth, Wi-Fi, NFC, ultrasound, etc.) communication with a remote device for telemetry, transferring data, transferring control information, receiving/sending feedback, etc. Any of these apparatuses may include one or more sensors, including electrical sensors (electrodes, etc.), pressure sensor(s), temperature sensor(s), optical sensor(s), etc., for detecting one or more physiological parameter. The sensor input may be used as feedback to the controller that may modulate or modify the apparatus, including one or more of: modifying a dose or does scheduling, triggering a dose, turning off or shortening a dose, lengthening a dose, increasing or decreasing the intensity of the applied light, modifying the frequency of the applied light, triggering an alarm or alert, triggering co-delivery of one or more agents, including sensitizing and/or desensitizing agents, or the like.

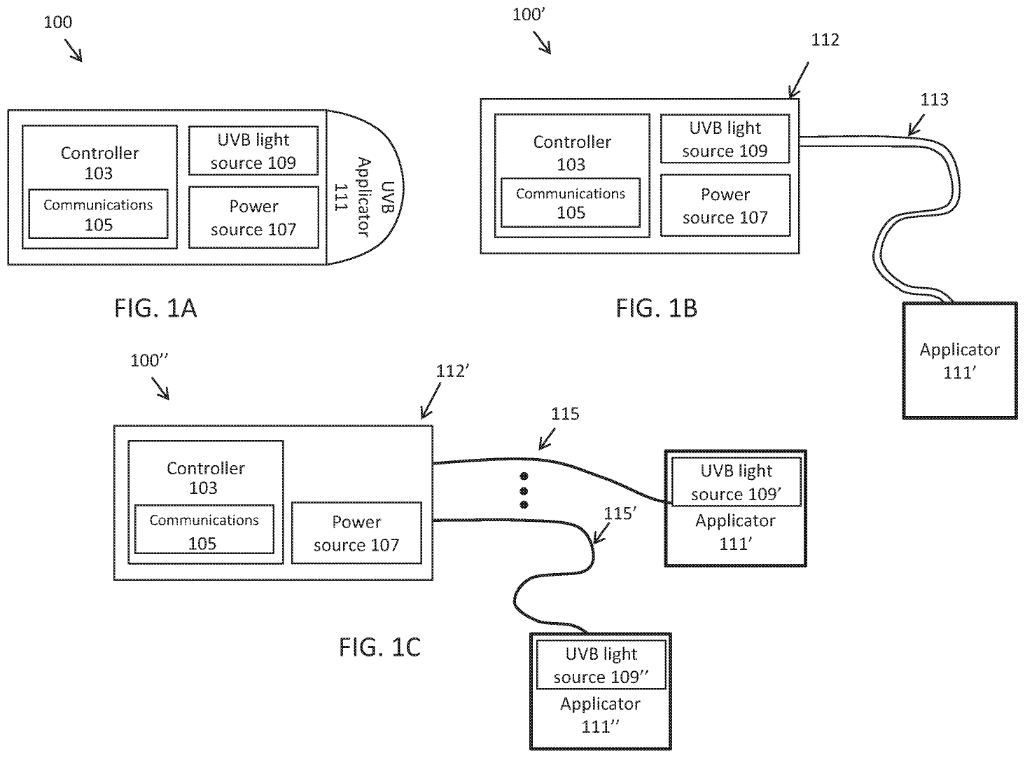

For example, FIG. 1A schematically illustrates a first example of an implantable apparatus 100 for delivering UVB light within a patient's body. In this example, the apparatus, is generally configured for implantation/insertion into a patient's body to deliver light (e.g., including but not limited to UVB light) to an internal tissue, such as a lymphatic system tissue. The implant includes a housing that may enclose a controller 103; the controller may include circuitry configured to control the delivery dose and/or dosing regimen of light (e.g., UVB light). This circuitry may include a processor, timer, memory, and the like. The controller may include or be connected to a communications module 105, which may wirelessly or directly receive and/or transmit information to/from a remote processor (including a smartphone, tablet, pad, etc.). The communications module may include one or more antennas and/or circuitry for wireless or wired communications (e.g., Bluetooth, ZigBee, near field, etc.). The apparatus typically includes a UVB light source 109 and/or other light source outside of the UVB range (including UVA light, UVC light, white light, etc.). The light source may be produced by any appropriate source, including light emitting diodes (LEDs) tuned and/or filtered within the desired light wavelength range(s), e.g., 290-320 nm, etc.), lasers, etc. Multiple light sources may be included. The intensity of the light applied may be adjusted by adjusting the power applied to the one or more light sources, including powering fewer light sources for lower powers, and multiple light sources for higher powers. The apparatus may also include a power source 107 which may be battery, including a rechargeable battery, or the like. In some variations the apparatus may be recharged by, e.g., inductive coils, which may be part of the communications 105 or separate therefrom. The apparatus shown in FIG. 1A also includes an integrated applicator (e.g., UVB applicator 111) for delivery of the light onto the tissue(s) of interest, such as a lymph duct or node, spleen, etc. The applicator 111 may include a lens for focusing/defocusing the light, one or more filters, etc. The applicator may also be referred to as the UVB emitter, as the location of emission of the UVB light to the target.

FIG. 1B is another example of an apparatus, similar to that shown in FIG. 1A, having one or more applicators 111′ that are connected to a base unit 112 (e.g., housing the controller, power source, light source(s), etc.) by a cabling 113; in this example, the cabling may include an optical waveguide (e.g., fiber optic) so that illumination generated by the base unit can be transmitted to one or more applicators (in FIG. 1B only one is shown, however, multiple applicators may be used) for delivery to the target tissue(s). The applicator may include a lens, filter or the like, or it may include a frame configured to secure the distal end of the applicator (which may be or include a bare end of an optical fiber) in proximity to the target tissue.

FIG. 1C shows another example of an apparatus 100″ having a base unit 112′ from which a plurality of connectors/leads (115, 115′) extend to connect with a plurality of applicators 111′, 111″. Although a plurality of applicators are shown, this apparatus may be configured with only a single applicator and/or lead. In this example, the applicator may be directly coupled with or include one or more light sources (e.g., UVB light sources 109′, 109″). In this example, the leads 115, 115′ may carry power to the light source(s), and do not need to transmit light; thus, these leads may be conductive leads (e.g., wires). The schematics of FIGS. 1A-1C are not shown to scale. Further, any of these apparatuses may include additional features, including a depot holding an agent (e.g. photosensitizing and/or desensitizing agent, etc.) that may be passively, tonically or controllably released, e.g., by the controller.

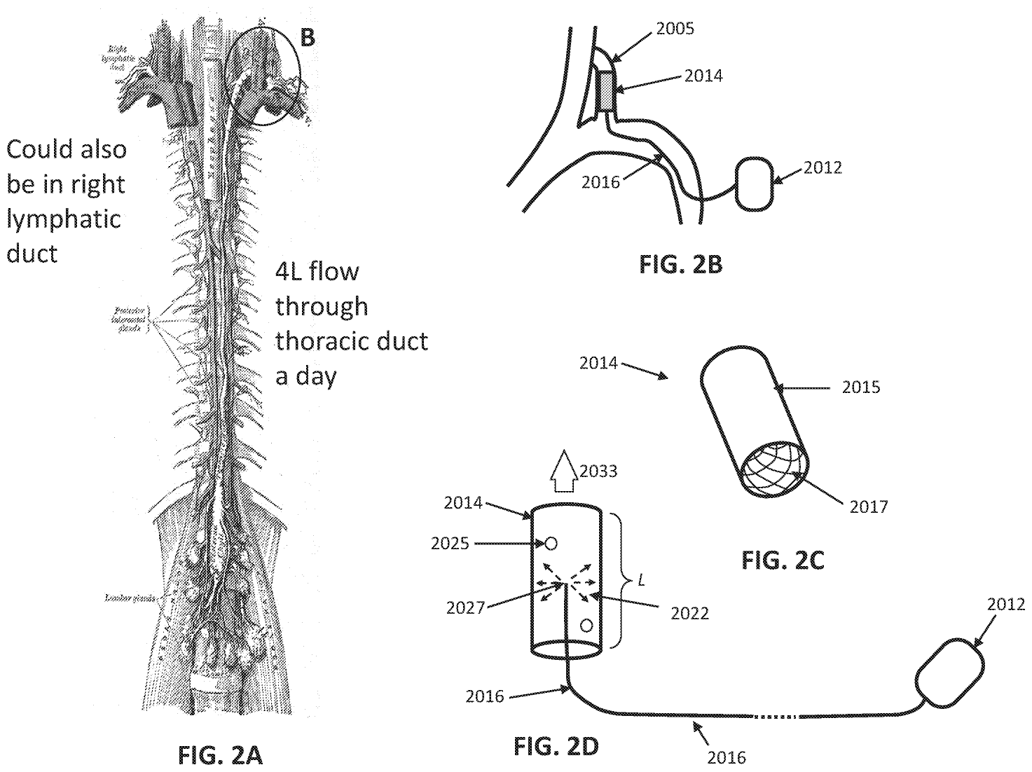

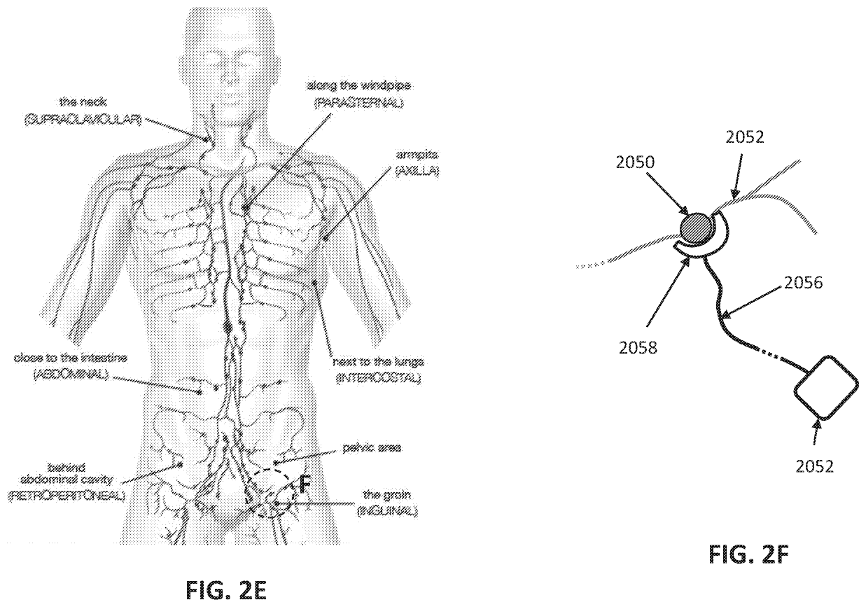

FIG. 2A schematically illustrates another example of an implantable apparatus for insertion into the patient's body to apply light therapy internally, e.g., on the lymphatic system. The system described in FIG. 2A includes a stent graft integrated with (e.g., forming part of) the applicator, or to which the applicator is attached. The stent has an expanding frame. The apparatus also includes a UV transmitting fiber-optic cable and a controller with a battery and LED. The stent graft could be placed, e.g., in a duct of the lymphatic system, such as the thoracic duct, lymphatic duct, lymphatic collecting vessel, cisterna chyli or bronchomediastinal trunk (illustrated in the left of FIG. 2A). The graft may be configured to expand to conform to the outer edges of the vessel with the UV light exiting perpendicular to the flow of fluid through the graft. The graft could be made of PTFE, ePTFE, woven PET, or other graft material. The graft material could be UV reflective, increasing the exposure of passing cells or could be coated in UV reflective coating. In an alternative configuration the LED light could be placed in the middle of the stent graft and instead of a fiber optic cable a power wire is connected to the LED. The expanding frame could be nitinol, stainless steel or any other somewhat rigid material that maintains the opening of the graft and the optical tip in the center of fluid flow. In another configuration, instead of a covered graft, the expanding frame, or stent, could be inserted without the graft material. The thoracic duct typically has approximately 4 L of flow each day and the total fluid in the lymphatic system is less than this, therefore, exposing the lymphatic system to UVB light directly may allow for the controlled suppression or apoptosis of the immune related cells such as B-Cells, T-Cells, Dendritic Cells (Dendritic Cells), Granulocytes, Lymphoid Cells, Megakaryocytes, Monocytes/Macrophages, Natural Killer Cells, and Thympocytes. UVB has may suppress or cause apoptosis of a broad array of immune cells important in disease related to immune system dysfunction. The range of light effective may include the UVB range, e.g., 280-320 nm. Although UVB therapy of the skin has been shown to be effective in the range of 300-315 nm, a broader range may be effective when the immune system is treated directly, bypassing the photoprotective effects of the skin. For this reason, light in the UVC range may also be effective (e.g., 100-280 nm). UVA, 320-400 nm, in conjunction with a photoactivating agent such as psoralen, coal tar, methotrexate, ciprofloxacin, ofloxacin, naladixic acid, azathioprine and venurafenib could also be used in conjunction with UVA light in order to cause apoptosis of cells. In this case, the patient may be locally or systemically administered a UVA photosensitizing drug and then the cells passing the UVA light would be treated, including but not limited to triggering apoptosis of these cells (allowing for transient cell destruction), dependent on the dose of light. The controller may cause the UV light to be turned on an off to scale up and down the immune suppression as needed. The controller could be implanted under the skin, allowing for wireless communication, programming and charging. The stent could be placed in a bypassed vessel to prevent occlusion of the vessel, such as a coronary artery bypass graft, either allograft or artificial. Feedback could be considered closed-loop with analytical or prescription algorithms applied to the controllers.

FIG. 2B illustrates the application of UVB/light therapy to the lymphatic system in another region of the body. In general, any region of the lymphatic system may be treated as described herein. The system described in FIG. 2B includes a UV transmitting fiber-optic cable and a controller with a battery and LED. Alternatively or additionally, the LED could be placed next to the lymph node and instead of a fiber optic cable, there would be a power cord to the controller instead. The controller could be implanted under the skin, allowing for wireless communication, programming and charging. The applicator of the system may include a diffuser around the lymph node to diffuse the UV light around the lymph node. The light used for immune suppression may be in the UVB range, e.g., 280-320 nm.

In general, the apparatuses described herein may provide a localized treatment region that nevertheless allows treatment of a large volume of biological material that passes through the localized region. In FIG. 2B-2D, the apparatus includes a frame 2014 configured as an approximately tubular stent or graft that can be inserted (and may self-expand) into a vessel of the body, such as the lymphatic duct 2005 shown in FIG. 2B. In this example a cable 2016 connects the frame to a driver 2012 that permits larger power supply, controller and in some variations the UVB light source, to be located distally from the frame.

The frame may insert into a body region such as the lumen of a vessel. In FIG. 2C, the frame is shown as a self-expanding frame 2017 made of a material such as Nitinol that is sufficiently elastic to contract and expand for both delivery and for moving with the body once inserted. The frame forms a chamber within which the light may be applied; the light may be limited to the region within the chamber that is formed. In FIG. 2C, the chamber is a channel formed as part of the lumen through the frame. To prevent light (e.g., UVB light) from escaping the side(s) of the channel, the walls (e.g., the cylindrical sidewall in this example) may include a UVB reflective or absorptive surface 2015 that prevents the light from laterally existing the treatment region (e.g., and illuminating the vessel), so that the UVB light illuminates the biological material passing through the channel of the frame but not laterally adjacent to the channel. For example, in FIG. 2C, the frame includes a reflective surface 2015 (reflective side facing the inner lumen of the channel; the outside surface, shown, does not have to be UVB reflective). The reflective surface may be a cover within or over the frame, or it may include a sheet of material within or over the frame. The reflective surface may be formed as part of the frame. Typically the reflective surface provides sufficient coverage to prevent light from passing out of the lateral sides of the passage. The reflective surface may be any appropriate material, including polymeric material (e.g., PTFE), having the desired optical properties.

Operation of the apparatus of FIGS. 2B-2C is shown in FIG. 2D. In FIG. 2D, the frame 2014 is inserted into a vessel in the patient's body (not shown) so that biological material (e.g., cells 2025, fluid, etc.) may pass through the channel in the frame, as shown by arrow 2033. The apparatus includes a UVB emitter 2027 mounted in the frame in a central location; in FIG. 2D, the emitter is mounted in the center of the channel (along the length, L, of the frame). The emitter may also be centered in the diameter of the channel and/or mounted to the side(s) of the channel. The emitter in this example is an end of a fiber optic line that extends (as cable 2016) to the driver 2012 (which may also be referred to as simply the controller) that includes a housing enclosing the power source, UVB light source, and controller circuitry (e.g., processor, communications circuitry, clock, memory, etc.). The UVB driver 2012 may generally drive the UVB dosing and may be implanted at a separate location from the applicator (e.g., UVB emitter and frame). Cells or other biological material (including fluids) passing through the channel will be irradiated by the UVB light when the apparatus is turned on; this light will be limited to the channel, avoiding any potential harm of exposure to the adjacent tissues, including the walls of the vessel into which the frame is implanted. Because a number of cells may pass through the channel during treatment, a large volume of target tissue (e.g., cells, including immune system cells) may be treated, despite having a very small footprint in the body.

FIGS. 2E and 2F illustrate another example of an apparatus similar to that shown in FIG. 2B-2D. In This example, the apparatus may be inserted in or around a localized region of the body, including in or around a localized region of the lymph system (shown in FIG. 2E). In FIG. 2F, the apparatus includes a frame forming an emitter housing 2058 that at least partially surrounds a lymph node 2050 or part of a lymph vessel 2052. The emitter housing include one or more (e.g., an array) of UVB emitters that may emit light in a controlled manner to treat the patient. For example, the frame may be a chamber having an inner surface (concave inner surface) that fits at least partially around a lymph node, as shown. The housing 2058 may be connected by a cable 2056, which may include fiber optics, forming or coupled to the emitters in the housing, that connects to a separate, implanted driver 2052, including a housing enclosing control circuitry, power source, and UVB light source.

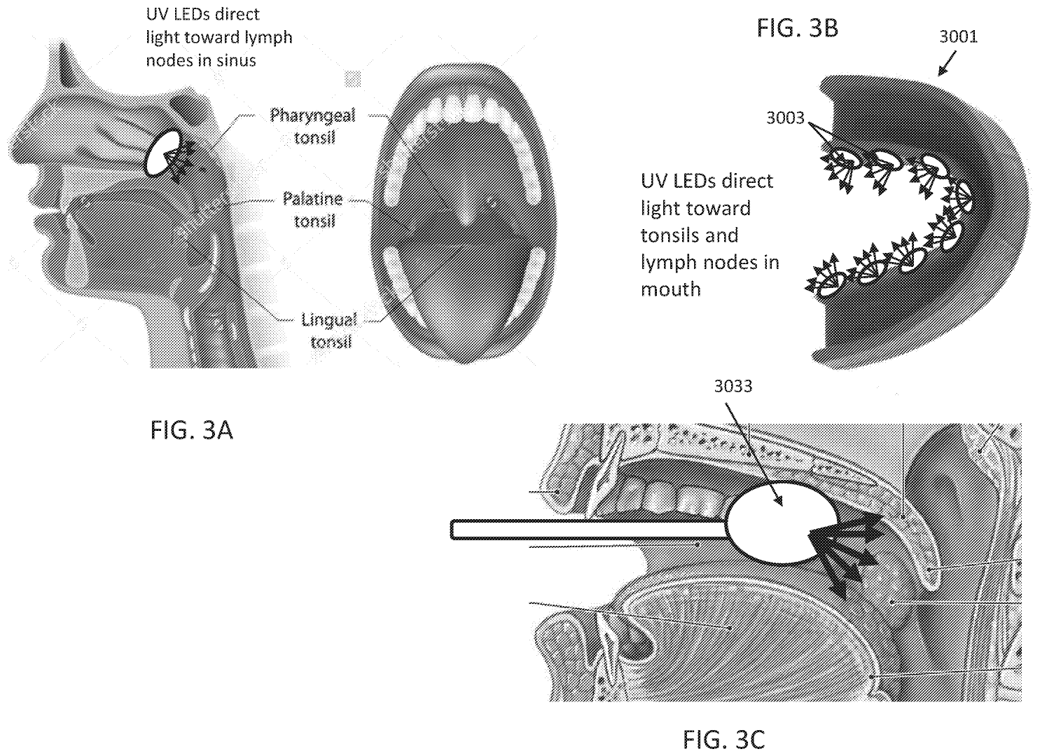

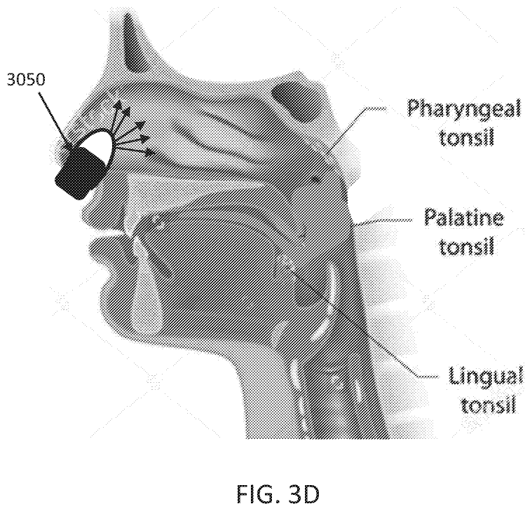

The systems describe in FIGS. 3A-3D may include a probe 3001 that transmits light to the lymphatic vessels in the mouth and/or nasal cavity, including the Pharyngeal, Palatine and Lingual tonsils. The probe could be inserted for treatment in any number of dosing regimens (daily, multiple days a week, monthly or annually) as needed. In FIG. 3B, for example, the UVB LEDs 3003 along with a power source and controller are placed in a mouthpiece worn by the patient and deliver UV therapy to the lymph vessels in the mouth and sinuses. This configuration could be worn all day or alternatively only at night. In some configurations, a plug may be is placed into the sinus with a controller, power source and UV LED that direct UV light from an emitter 3033 to the lymph nodes and vessels in the sinus, as shown in FIG. 3A. In FIG. 3C, the UV light could be generated with a non-LED light source such as a compact fluorescent lamp, excimer laser or other UV generating light source, and applied in the back of the subject's mouth. In FIG. 3D, the apparatus 3050 may include a nose-plug body adapted to fit into and be secured within one or both nostrils, and may include a light for applying illumination and/or a controller for regulating the application of the (e.g., UVB) light) as described herein. The nose plug body portion may partially sit outside the nose for attachment and can be easily remove, it may include an external projection. Such light sources may be adapted for use with any of the variations described herein. Any of these apparatuses, including those shown in FIGS. 3A-3D, may be used to treat one or more conditions, such as allergic rhinitis, allergic spasm in esophageal structures, some food allergies, etc.

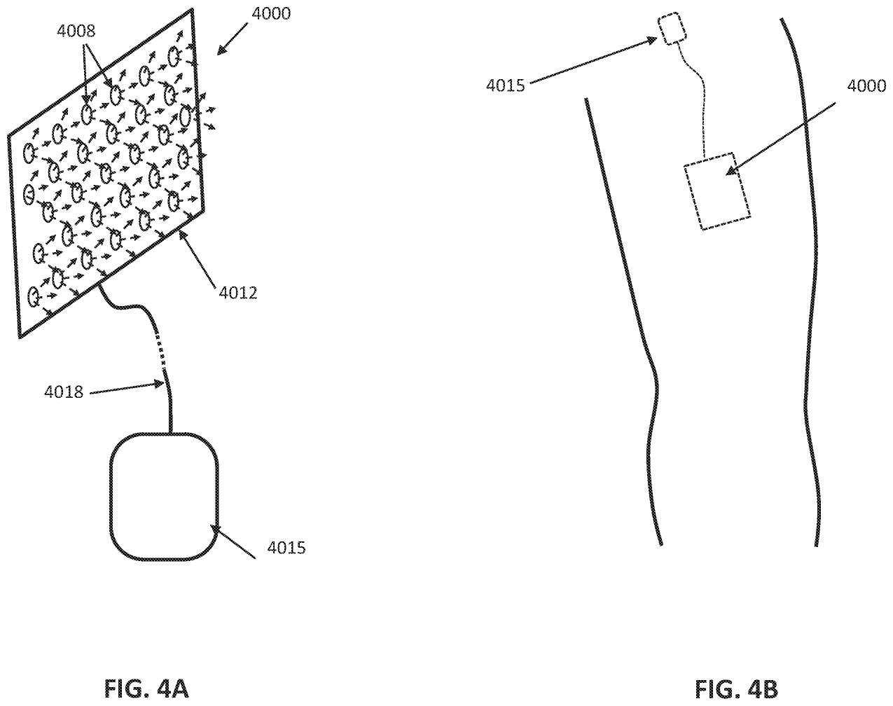

FIGS. 4A and 4B illustrate implantable UVB light applicators 4000 that may be subdermally applied under the skin. The system described in FIG. 4A includes a cable 4018 (e.g., optical and/or power line), an array of emitters 4008, which may be, e.g., the exposed regions of a fiber optic or LED lights (UV emitting LEDs), and a driver 4015 (e.g., controller, a battery, etc. within a housing). The driver could be implanted under the skin, allowing for wireless communication, programming and charging. The applicator may include a diffuser around array to diffuse the UV light. Alternatively or additionally, a wave guide or OLED could be used to generate and or distribute the light. The LED array could be placed on a thin flexible substrate to allow for the array to flex and conform to the local anatomy skin. In an alternative configuration, the apparatus may include a string of LEDs that run parallel to the lymph nodes when inserted.

In FIG. 4A, the array of emitters 4008 are arranged on a thin and flexible substrate 4012. The substrate is UVB reflective or blocking, and the emitters are located on just one side of the substrate. This may allow the device to emit light in one direction (e.g., towards a target tissue) while protecting the adjacent tissue. The substrate may be rolled into a tube (or partial tube) or may be implanted in a way that conforms to the target tissue. For example, as shown in FIG. 4B, the apparatus 4000 of FIG. 4A may be implanted under the patient's skin; the driver 4015 portion may be separately implanted (or implanted in a separate location), and may be connected to multiple emitters.

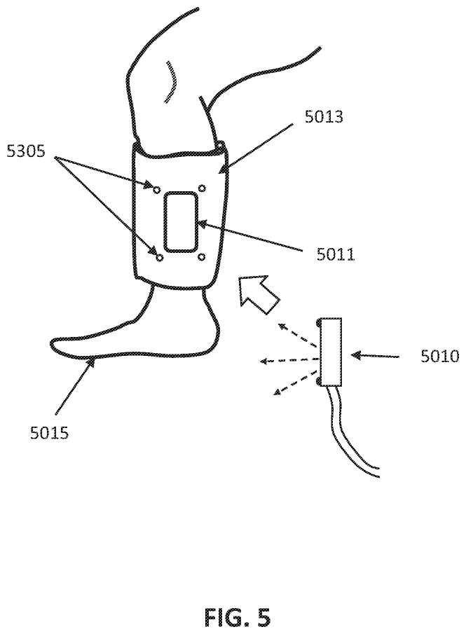

As mentioned, in any of the variations described herein one or more photosensitizing agents may be administered in conjunction with the internal light therapy. For example, the system described in FIG. 5 includes an occlusive dressing 5013 (e.g., hydrocolloid dressing) with a temporary attachment method to a UV light source. In FIG. 5, the attachment mechanism is a plurality of magnets that can secure the applicator 5010 to the window (UV transparent window 5011) of the dressing. The occlusive dressing could be constructed to deliver a medicament such as coal tar, psoralen, steroid, salicylic acid, vitamin D analogue, etc. The section of the occlusive dressing used to deliver UV light may be constructed with material that is UV transparent and may be constructed to not have medicament in this section. The occlusive dressing would be a wrap, Velcro strap, hydrogel, hydrocolloid or other material used in creating occlusive dressings. It is known that immunosuppressive effects of UV light therapy may spread beyond the treatment area to the surrounding tissue or systemically, therefore this system of occlusive dressing and UV light may provide for treatment of a skin condition such as psoriasis, vitiligo, dermatitis, etc. beyond the area specifically treated with light therapy. In another configuration, the area treated with light may be periodically moved to allow for exposure of other areas of the skin with the UV light. In FIG. 5, the dressing is worn on the leg of a patient having an inflammatory disorder such as psoriasis 5015.

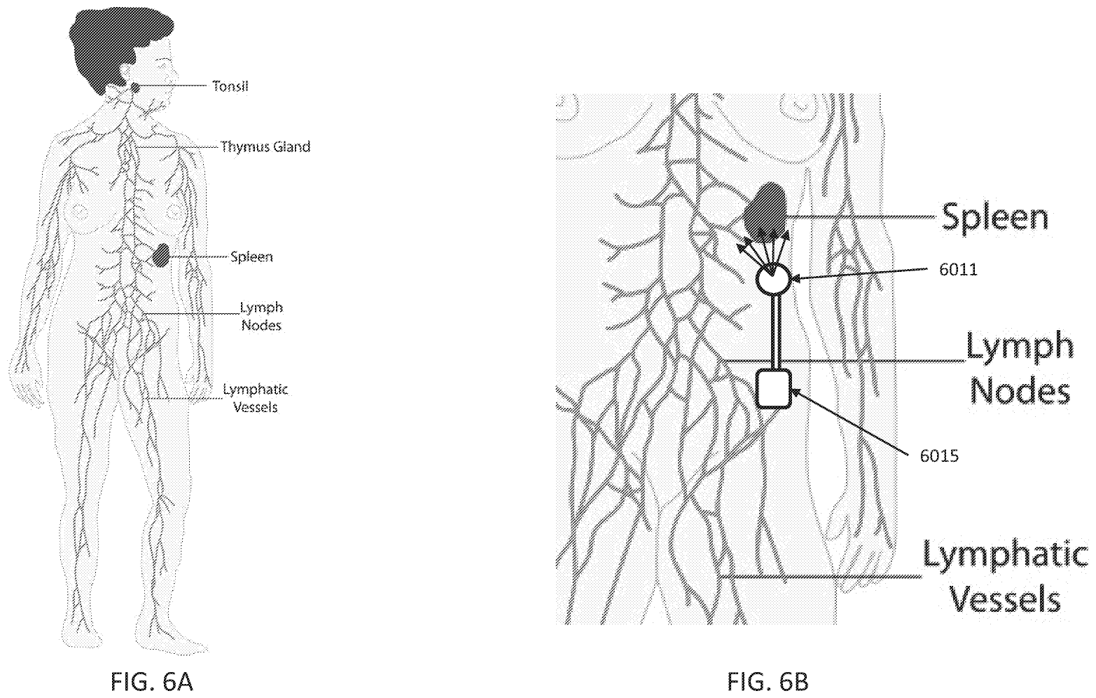

FIGS. 6A and 6B illustrate the use of another example of an apparatus as described herein for use in delivering for UVB light. In the system shows schematically in FIG. 6B a UV LED light source 6011 is connected to a driver 6015 including a housing containing a controller and power source. This system could be implanted next to an organ in the body, such as the spleen or thymus, know to generate immune cells. In addition, it could be implanted next to a transplanted organ to reduce the immune response to the transplanted organ or could be implanted in an organ such as the heart, lung, liver, kidney, pancreas, intestines, skin graft, stomach, testis, hand, coma, islets of Langerhans, or heart valve.

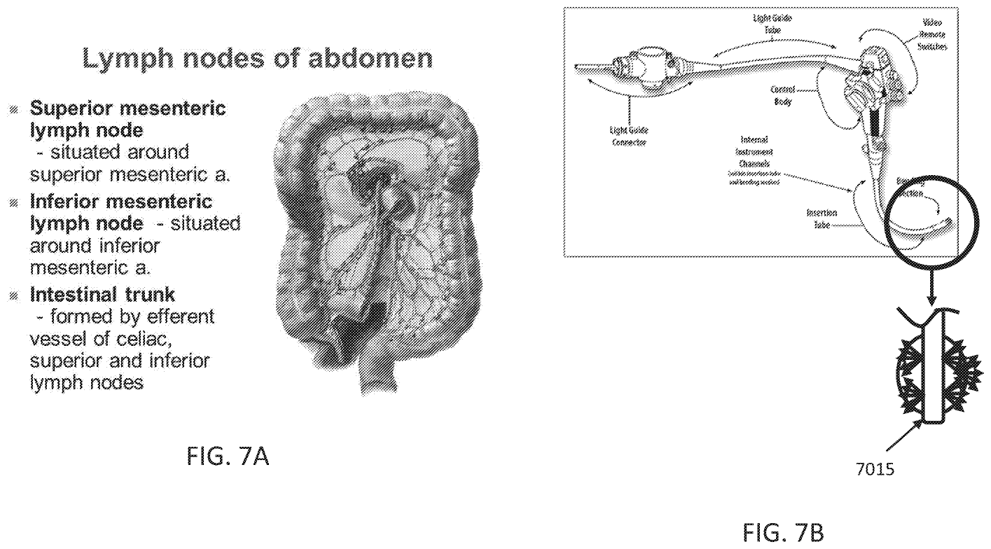

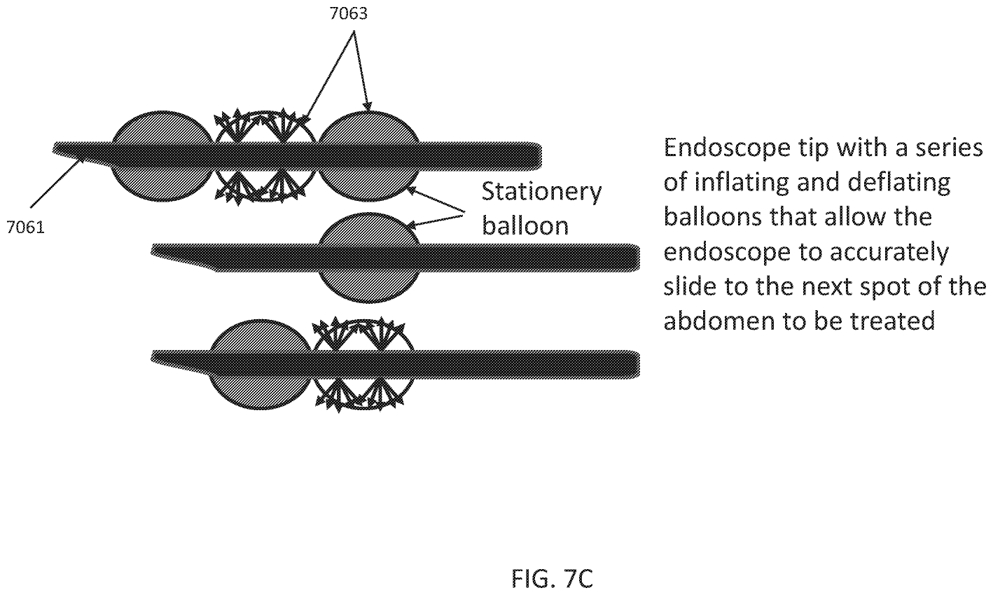

The system described in FIG. 7B may be used to treat one or more lymph nodes from the abdomen (as illustrated in FIG. 7A, showing typical anatomy). In FIG. 7B, the apparatus may include an endoscope with a tip 715 that has UV LEDs to deliver UV light to the walls of the abdomen. In one alternative, the LEDs could be contained within a water filled balloon to allow for equal distribution of light to the walls of the intestines and adjacent structures. The endoscope tip with an air- or water-filled balloon may provide good contact with abdominal wall and UVB light shining out of the balloons for treatment of the abdominal walls and adjacent structures.

The balloon would be inflated in order to stabilize the position in the stomach. In an alternative embodiment (e.g., FIG. 7C), multiple balloons could be inflated in order to move along the length of the intestines, ensuring that a precise dose is given to the entire length of the intestines. In FIG. 7C, the endoscope tip 7061 includes a series of inflating and deflating balloons 7063 that may allow the endoscope to accurately slide to another portion of the abdomen for treatment. The endoscope may be used to directly treat the structures of the abdomen including the stomach, intestines, lymph nodes and vessels. This type of treatment could be used to decrease the immune response for diseases such as lupus, IBD, MS, Chrohns, Guilliane-Barre & CIDP, Graves, Myasthenis Gravis and may include immune modulated diseases of the skin such as psoriasis, dermatitis, vitiligo, etc.

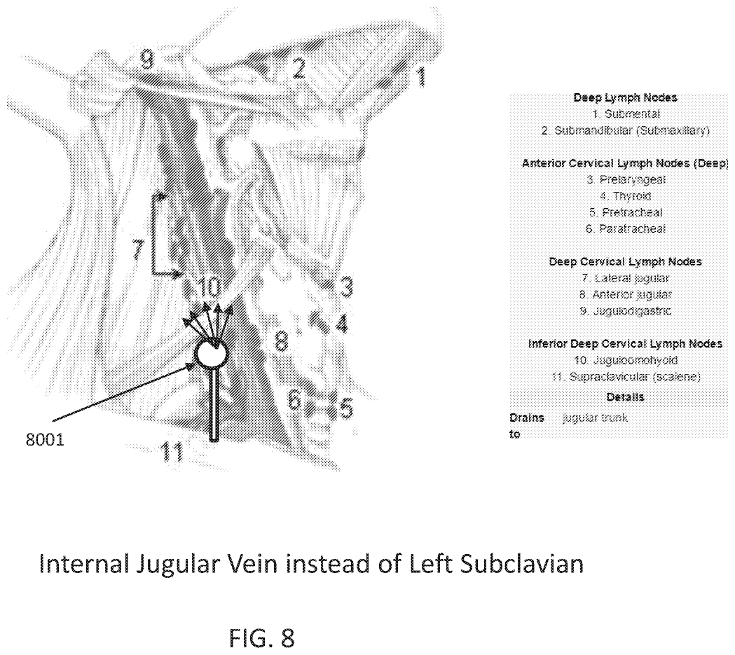

The system (apparatus 8001) described in FIG. 8 is similar to the systems described in FIGS. 2A, 2B and 6B, above, except in this example the apparatus is adapted for treatment of the lymphatic nodes and vessels of the central nervous system (CNS). This could be used to treat immune related diseases of the CNS such as multiple sclerosis. It may also be used to treat mental disorders that may have an immune system related component such as depression, bipolar disorder or schizophrenia. The device may be sized, powered, positioned and otherwise configured for such treatment.

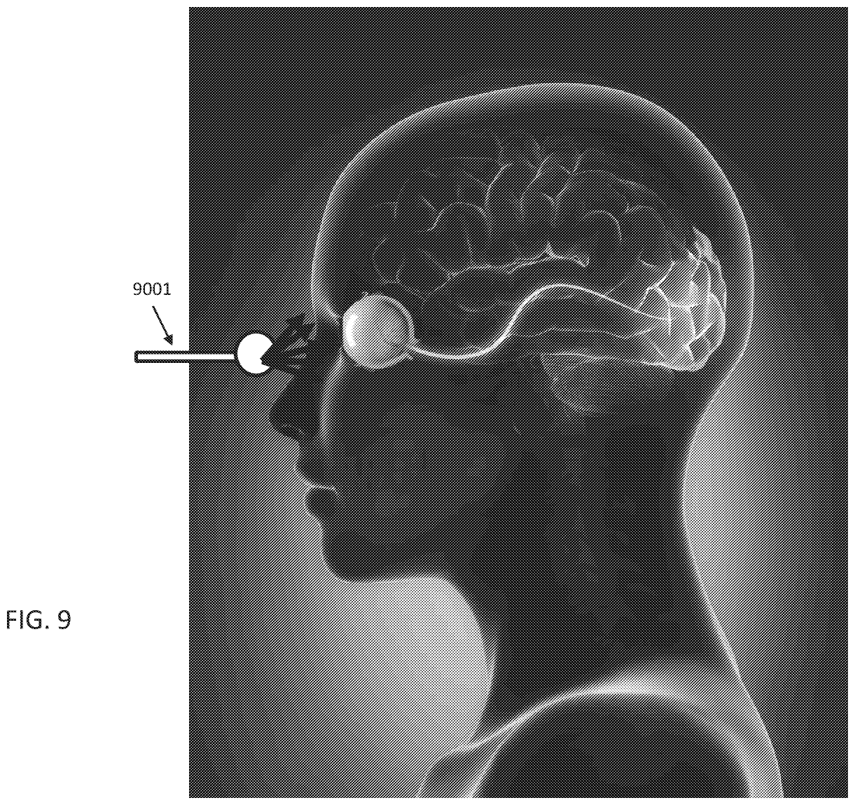

FIG. 9 gives a schematic overview of an apparatus 9001 configured to deliver therapy to or through a patient's eyes, e.g., by including UV LEDs that direct light towards the optic nerve. The apparatus described in FIG. 9 may be similar to the system shown in FIGS. 3A-3D or FIG. 4A-4B, but directed towards the optic nerve, which may aid in the treatment of diseases such as MS. In this example, the light may be directed away from the UV sensitive tissues of the eye to prevent damage to these areas.

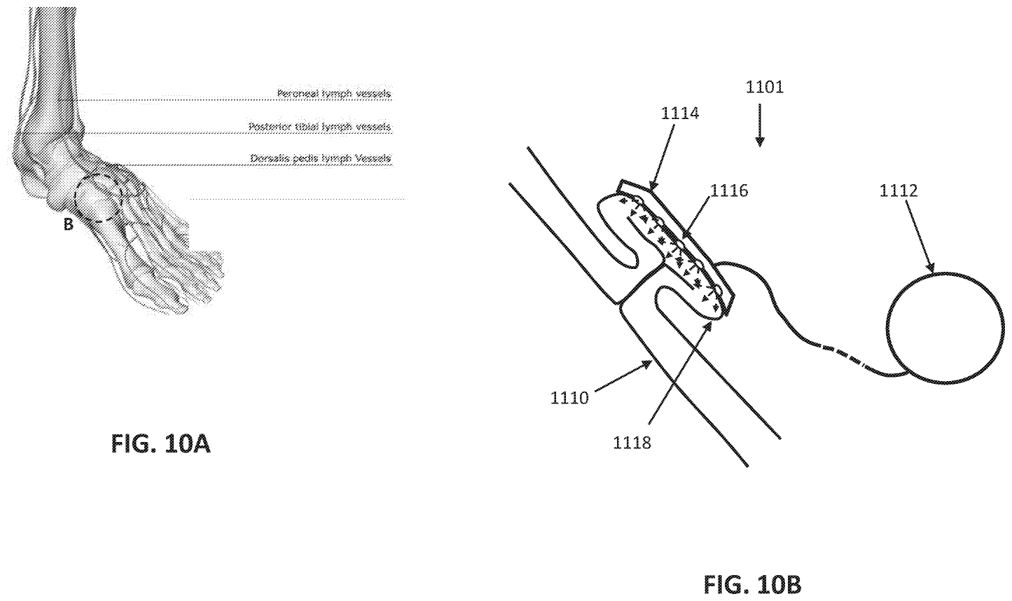

The system 1101 described in FIG. 10B is adapted for use with a shunt 1118 formed in a patient's body. In FIG. 10B, the apparatus 1101 connects on, in or adjacent to the shunt, and including a plurality (e.g., an array) of UV (e.g., UVB) emitters 1116 on a housing or substrate 1114, and a driver 1112 controller with a power source. The driver 1112 could be implanted under the skin, allowing for wireless communication, programming and charging. The shunt configuration allows for UV treatment of lymphatic fluid in a lymph vessel 1110, thereby systemically suppressing the immune response. Various vessels in the lymphatic system could be shunted such as thoracic duct, lymphatic duct, cisterna chyli, lymph vessels in the foot or bronchomediastinal trunk.

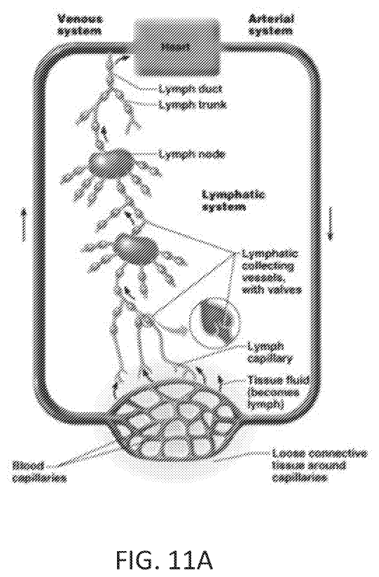

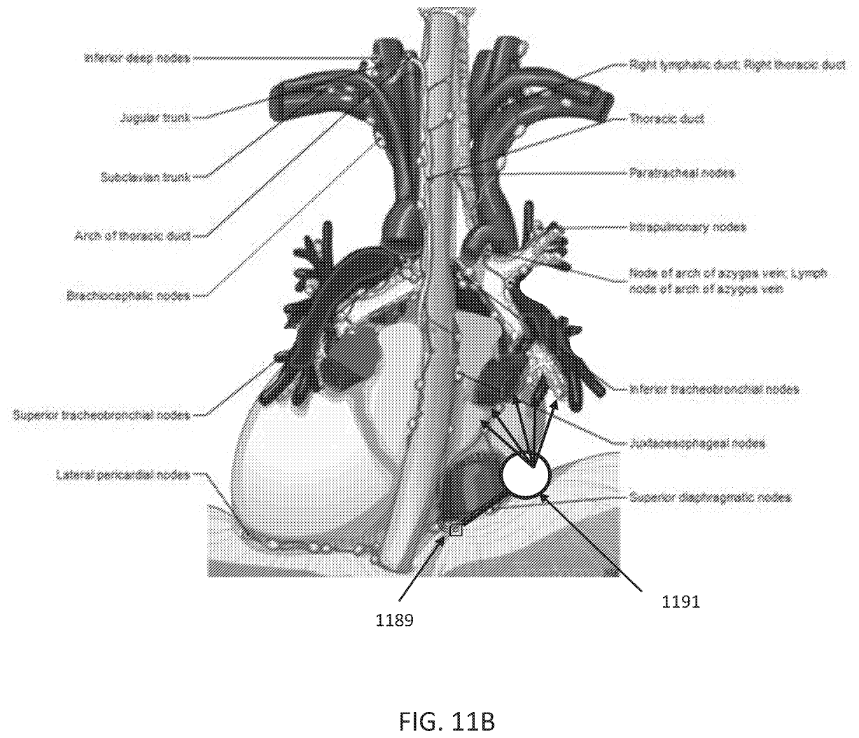

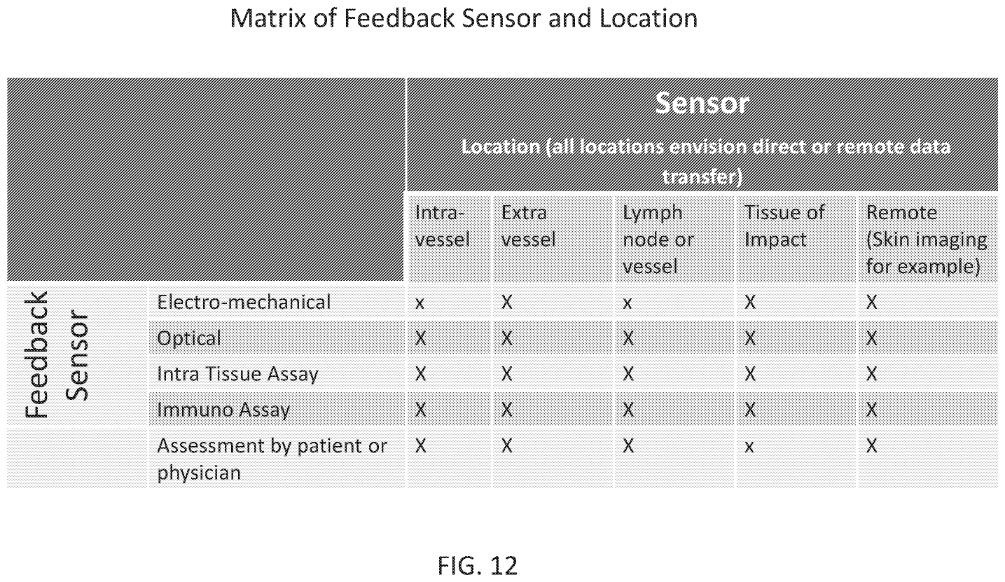

FIG. 11A is an overview of the lymphatic vessels that may be treated as described herein. In FIG. 11B, a system (similar to that described in FIGS. 2A-2B) is shown configured to include feedback. In FIG. 11B, the apparatus has a sensor 1189 (e.g., implanted near, adjacent or next to UV light source 1191) that provides for a feedback loop to increase or decrease the amount of light that is delivered to the lymph node (in this example, a lymph node in/on the heart). As described in FIG. 12, this sensor could be located in a number of places, such as intra-vessel, extra-vessel, lymph node or vessel, tissue receiving the light or outside the body. The sensor providing feedback could be electro-mechanical, optical, intra tissue assay, immune assay or be physician or patient assessment. If the implant was used to treat a specific disease, the symptoms of the patient could be used to increase or decrease the dose of light. Lymphatic vessels collect lymph from lymph capillaries, deliver lymph to lymph nodes, and return fluid to circulatory veins near the heart. The right lymphatic duct and thoracic duct may be targets for the apparatuses described herein.

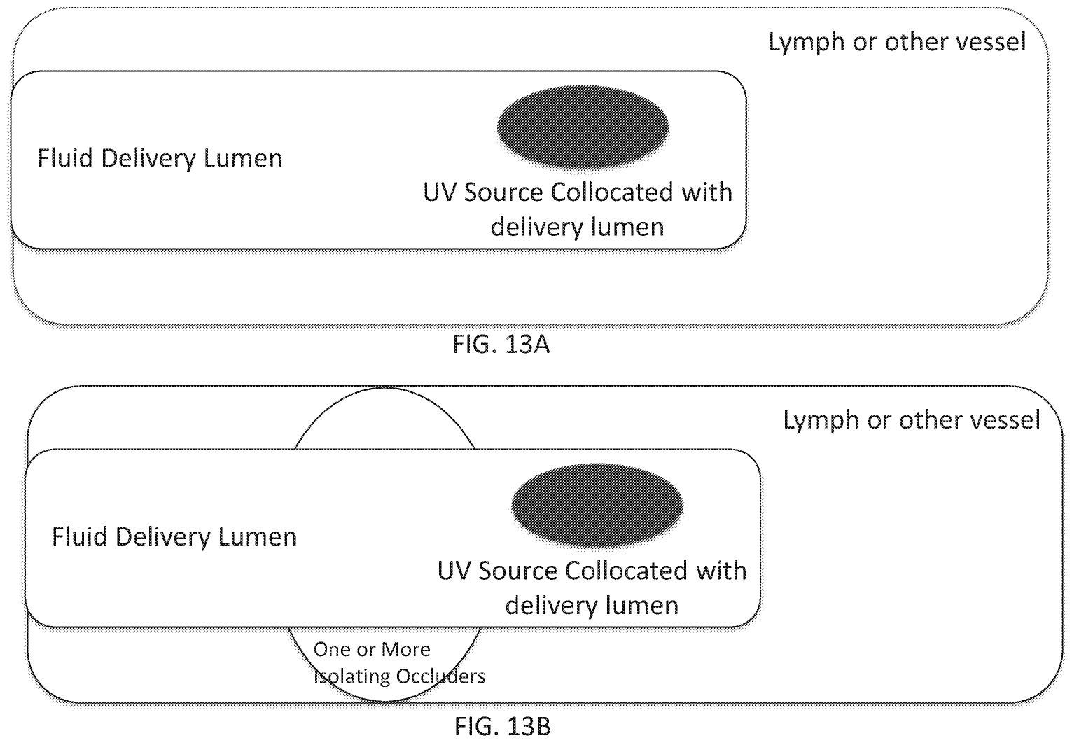

In general, any of the apparatuses described herein may include treatment by delivery of UVB light by applying effective doses of UV light. UV light may be applied continuously, or in a non-continuous (e.g., pulsing, period, etc.) matter. Further, the intensity of the UV light may be constant or varying. The intensity may be within a predetermined range that is effective for internal treatment. Finally, the location of the UVB light maybe adjusted for appropriate treatment. The system described in FIGS. 13A-13B includes a light source delivered in combination with a local fluid/drug. Alternatively or additionally, a systemic drug or medicament may be used. The drug may or may not be photoactive to UV. For example, UVA, 320-400 nm, in conjunction with a photoactivating agent such as psoralen, coal tar, methotrexate, ciprofloxacin, ofloxacin, naladixic acid, azathioprine and venurafenib could be used in conjunction with UVA light in order to cause apoptosis of cells. In this case, the patient would be locally administered a UVA photosensitizing drug and then one or more isolating occluders could temporarily stop fluid flow, and local cells would be apoptosed, allowing for transient cell destruction, dependent on the dose of light.

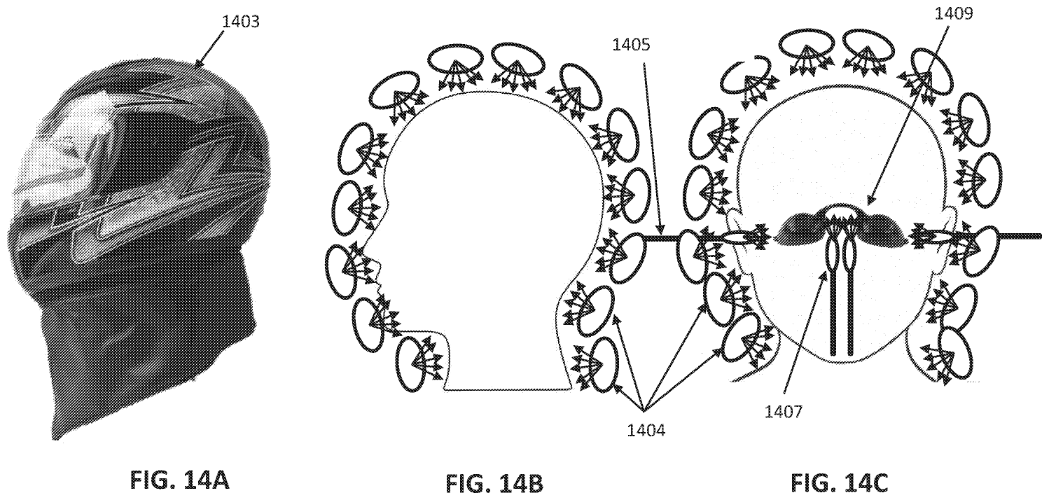

In addition to the methods and apparatuses described herein, externally applied light may be applied in addition or instead of the internally-applied light described herein. For example, FIGS. 14A-14C illustrates one example of a system that includes a helmet 1403 that extends onto the neck and a plurality of emitters 1404 (and one or more UV light source(s)) that lights the entire surface of the helmet wearer with UV light. There may also be additional probes to light the inner ear 1405, nose and mouth 1407 and googles 1409 to protect the eyes. There may also be light guides to help direct light to the scalp and past the hair.

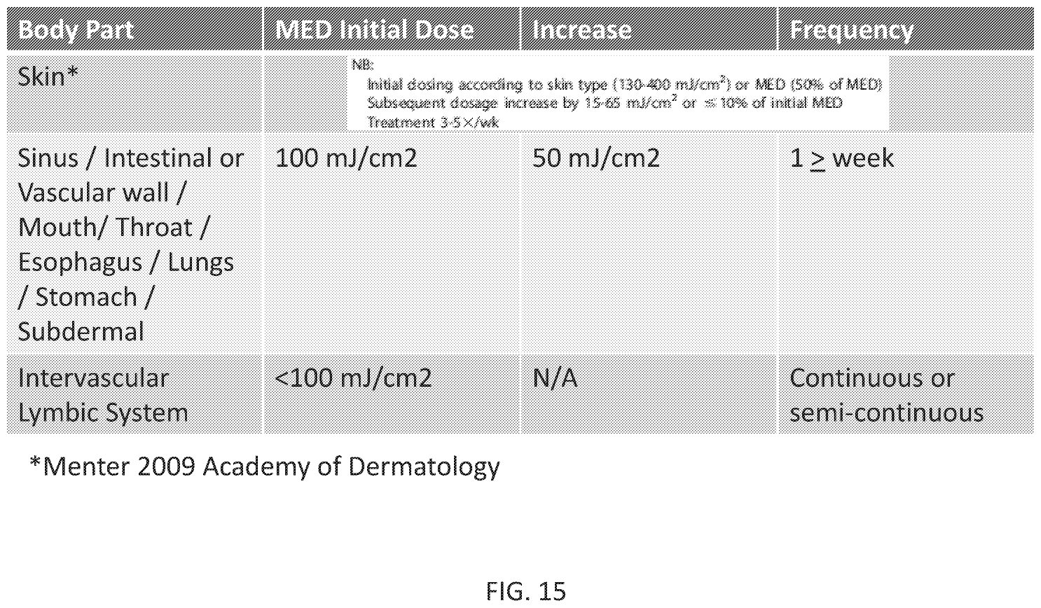

FIG. 15 shows a table that estimates the dosing and frequency of dose for treatment specific dosing of UVB light. In general, any of the apparatuses described herein may include treatment by delivery of UV light by applying effective doses of UV light. Dose is defined by energy over an area, for example, mJ/cm2. The intention of the light is to apoptosis and de-activate immune cell without causing significant irritation or damage to adjacent structures. In the skin, this is called the minimal erythema dose or MED. It is unknown whether internal structures in the body have a photo-adaptation response to UV light like the skin, but it is likely that there is variation person to person on the maximum dose that can be delivered without causing a significant damage or irritation in the surrounding tissue. This table cites the American Academy of Dermatology phototherapy guidelines (Chapter 5, 2009) for treatment of psoriasis to determine skin treatment. It is expected that tissue without an epidermis will respond to lower doses of light, in one study patients with Oral Lichen Planus responded to doses between 100-400 mJ/cm2 once a week. If devices are applied in the limbic system, it is expected that they may be treating limbic fluid as it passes by the light source, therefore it will have continuous or semi-continuous dosing to treat the immune cells as they pass by the light source. A continuous on or semi-continuous on light source may have a lower radiance on the immune cells (<100 mJ/cm2) but stay on for several hours or days to effectively systemically suppress the immune system. This is because the cells would be in the range of the light source for only a brief period of time but since the light source would be on for an extended period, the dose would be cumulative as the cells passed by multiple times during the course of lymphatic fluid circulation.

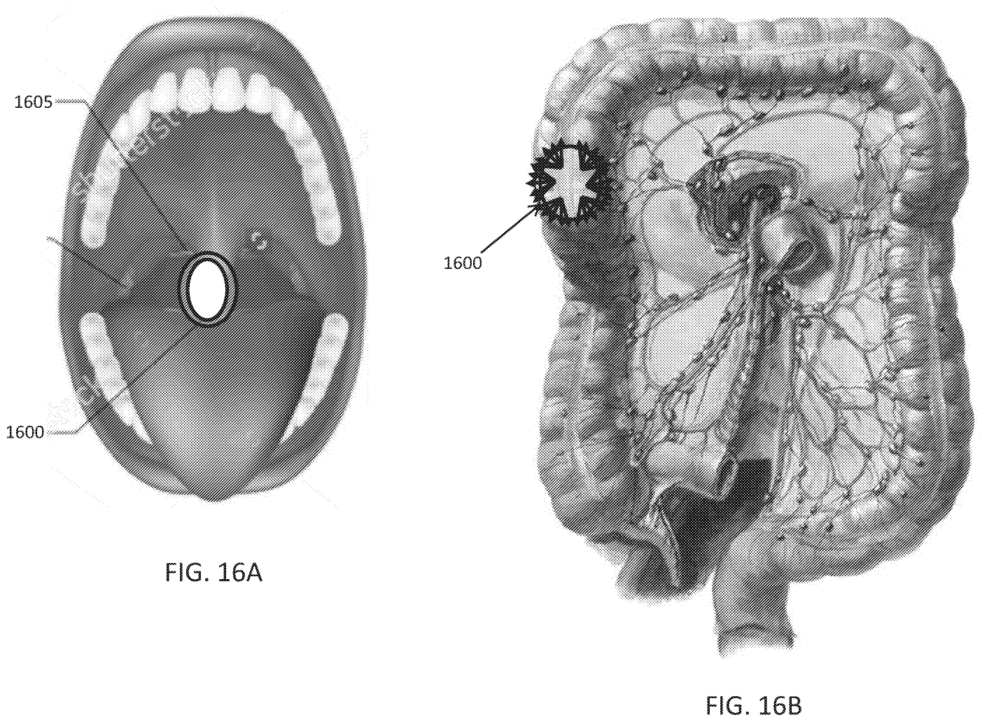

In FIGS. 16A and 16B, one variation of an apparatus as described herein is illustrated, configured as a pill 1600. The pill form emits UV light (e.g., UVB) from all sides 1605 of the pill (e.g., may include multiple UVB emitting photodiodes around the perimeter of the pill). The pill apparatus may be swallowed and then turned for a predetermined time period on to deliver UV light within the digestive track or any sub-region(s) thereof (e.g., throat, esophagus, stomach, large intestine, small intestine, etc.). In addition, the pill could be combined with optional drug delivery and programmed to turn on an off in specific intervals in order to aid with the distribution of the dose throughout the abdomen. The light or drug delivery could be turned on or off when the pill senses it is in a certain location. For example, it could turn on or deliver drugs when it detects acid in the stomach or turn on when it detects pressure from the stricture of the abdominal wall. The control of turning it on or off could occur outside the body as well through electronic communication and the pill could record data or images from the digestive system as it is traversing through it. This data could be delivered electronically and this data could be used to determine when to turn it on or off. The data could include, heat, temperature, pH, moisture levels, pressure, location, optical images and video, etc.

As mentioned, the UV light (e.g., UVB light) may be applied continuously, or in a non-continuous (e.g., pulsing, period, etc.) matter. Further, the intensity of the UV light may be constant or varying. The intensity may be within a predetermined range that is effective for internal treatment. Finally, the location of the UVB light maybe adjusted for appropriate treatment. In some variations, the light may be applied for a dose of between 0.01 second and about 1 hour. For example, the dose duration may be applied for between 0.01 second and about x seconds, where x is 0.1 seconds, 0.2 seconds, 0.3 seconds, 0.4 seconds, 0.5 seconds, 0.6 seconds, 0.7 seconds, 0.8 seconds, 0.9 seconds, 1 second, 2 seconds, 3 seconds, 4 seconds, 5 seconds, 6 seconds, 7 seconds, 8 seconds, 9 seconds, 10 seconds, 12 seconds, 15 seconds, 20 seconds, 25 seconds, 30 seconds, 35 seconds, 40 seconds, 45 seconds, 50 seconds, 55 seconds, 60 seconds, 2 minutes, 4 minutes, 5 minutes, 6 minutes, 8 minutes, 10 minutes, 12 minutes, 15 minutes, 20 minutes, 25 minutes, 30 minutes, 35 minutes, 40 minutes, 45 minutes, 50 minutes, 55 minutes, 1 hour, etc. For examples, doses having a duration of between about 0.01 second and 2 seconds, between about 0.01 seconds and 5 seconds, between about 0.1 seconds and 10 seconds may be preferred, between about 1 second and 1 minute may be preferred, etc.

As mentioned, the dose may be continuous or periodic, including applied at an on/off frequency of between 10 kHz and 1 Hz, such as between about 10 kHz and 1 kHz, between about 1 kHz and 0.1 kHz, between about 1 kHz and 0.5 KHz, between about 1 kHz and 10 Hz, etc.

In addition, the dose may be repeatedly applied, e.g., y times per hour, day or week (where y is between 1 and 100, e.g., y is 1, 2, 3, 4, 5, 6, 7, 8, 9, 10, 11, 12, 15, 20, 25, 30, 35, 40, 45, 50, 55, 60, etc.). For example, a dosing regimen may include applying light for a duration of about x seconds or more, repeated at least y times per day.

The strength or power of the light applied (which may depend upon distance from the light emitter, typically very close in an implantable system, such as <0.1 mm away) may be estimated as the intensity (e.g., W/m²) or as the radiance. The dose strength may be estimated as the intensity multiplied by the duration (time) (e.g., millijoules/sec*cm² times duration, giving mJ/cm²). The strength of the applied light may be referred to in relation to the light source, for example, as mW/cm² of UVB light emitted. The intensity of the internally applied light sources described herein may be relatively low (e.g., between 0.001 mW/cm² and 10 mW/cm², between 0.01 mW/cm² and 1 mW/cm², between 0.001 mW/cm² and 1 mW/cm², between 0.001 mW/cm² and 0.1 mW/cm², etc.).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-87. (canceled)

88. An implantable apparatus for applying ultraviolet (UV) illumination in a contained manner within a patient's body, the apparatus comprising:
an expandable frame having a lumen forming a channel for passing a biological material;
a UV emitter within the channel;
a UV driver coupled to the UV emitter, wherein the UV driver includes a power source and a controller to control UV emission from the UV emitter; and
a UV reflective or absorptive surface in or around the channel, wherein the UV reflective or absorptive surface is configured to block or reflect UV light from the UV emitter, so that the UV light illuminates the biological material passing through the channel but not laterally adjacent to the channel.

89. The apparatus of claim 88, wherein the UV emitter comprise a fiber optic.

90. The apparatus of claim 88, wherein the UV emitter is coupled to the side of the channel.

91. The apparatus of claim 88, wherein the UV emitter is mounted in a central region of the channel.

92. The apparatus of claim 88, further comprising a cord extending between the UV driver and the UV emitter.

93. The apparatus of claim 92, wherein the cord comprises a fiber optic cable, further wherein the UV driver comprises a UV light source.

94. The apparatus of claim 88, wherein the UV driver comprise a housing enclosing the power source and the controller.

95. The apparatus of claim 88, wherein the power source comprises a rechargeable battery.

96. The apparatus of claim 88, wherein the controller comprises a microcontroller.

97. The apparatus of claim 88, wherein the UV reflective or absorptive surface comprises a reflective surface within the channel.

98. An implantable apparatus for applying ultraviolet B (UVB) illumination in a contained manner within a patient's body, the apparatus comprising:
an expandable frame having a lumen forming a channel for passing a biological material;
a UVB emitter within the channel;
a UVB driver coupled to the UVB emitter, wherein the UVB driver includes a power source and a controller to control UVB emission from the UVB emitter within the UVB range of 290 to 320 nm; and
a UVB reflective or absorptive surface in or around the channel, wherein the UVB reflective or absorptive surface has a reflective inner surface and is configured to block or reflect UVB light from the UVB emitter within the channel, so that the UVB light illuminates the biological material passing through the channel but not laterally adjacent to the channel of the device.

99. A method of applying ultraviolet (UV) illumination in a contained manner within a patient's body, the method comprising:
turning on a UV emitter that is positioned within a lumen forming a channel through a frame, wherein the frame is implanted into a lumen of a vessel in the patient's body proximate to a lymph node; and
emitting light from the UV emitter to irradiate biological material passing through the lumen through the channel.

100. The method of claim 99, further comprising absorbing or reflecting UV light from a sidewall of the channel to prevent irradiation of a region of the lumen of the vessel that is laterally adjacent to the channel.

101. The method of claim 99, further comprising inserting the frame within the lumen of vessel in the patient's body so that biological fluid passes through the channel.

102. The method of claim 101, wherein inserting further comprises allowing the frame to self-expand in the lumen of the vessel in the patient's body.

103. The method of claim 99, further comprising turning off the UV emitter after delivering a dose of between 0.01 seconds and 10 seconds.

104. The method of claim 103, further comprising applying multiple doses of the UV light by repeating the steps of turning on, emitting light and absorbing or reflecting light at a dose frequency of between 1 and 200 doses/day.

105. The method of claim 99, wherein turning on comprises controlling, by an implanted controller, power delivered to the UV emitter.

106. The method of claim 99, wherein emitting light from the UV emitter comprises emitting light from a fiber optic having a distal end terminating within a lumen of the channel formed through the frame.
